(19) United States
(12) Patent Application Publication
(10) Pub. No.: US 2012/0108905 A1
(43) Pub. Date: May 3, 2012

(54) PORTAL APPARATUS WITH A CONTOURED COMPLIANT SEAL

(75) Inventors: Sally Carter, Wallingford, CT (US); Andrew Hudon, Superior, CO (US); Andrew Barnes, Naugatuck, CT (US)

(73) Assignee: Tyco Healthcare Group LP

(21) Appl. No.: 13/221,006

(22) Filed: Aug. 30, 2011

Related U.S. Application Data
(60) Provisional application No. 61/407,581, filed on Oct. 28, 2010.

(51) Int. Cl. A61B 1/32

(52) U.S. Cl. 600/208

(57) ABSTRACT

The present disclosure relates to a contoured instrument seal for arthroscopic, laparoscopic and endoscopic surgery. The contoured instrument seal includes a body, an annular inner edge, an annular outer edge, and one or more raised concentric rings, e.g., having a spiral shape. The body includes a top surface and a bottom surface. The annular inner edge defines an opening adapted to receive a surgical instrument. The annular outer edge may be adapted to mechanically mount to an inner portion of a seal housing. The raised concentric ring is defined on a surface of the resilient body between the annular inner edge and the annular outer edge.

### CROSS-REFERENCE TO RELATED APPLICATIONS

This application claims priority to, and the benefit of, U.S. Provisional Patent Application Ser. No. 61/407,581, filed on Oct. 28, 2010, the entire contents of which are hereby incorporated by reference herein.

### BACKGROUND

1. Technical Field

The present disclosure relates to an instrument seal adapted to mechanically couple to a portal apparatus. In particular, the present disclosure is directed to a contoured compliant instrument seal adapted to establish a substantial sealed relation with a surgical instrument during a surgical procedure.

2. Description of the Related Art

Arthroscopic, laparoscopic and endoscopic procedures generally require that any surgical instrument inserted into the body be sealed. In other words, gases and fluids should not enter or exit the body through an incision as, for example, in surgical procedures in which the surgical region is insufflated or provided with saline solution. Moreover, laparoscopic and endoscopic procedures often require a clinician to treat organs, tissues, and vessels far removed from the incision, thereby requiring surgical instruments used in such procedures be relatively long and narrow.

Typically, a surgical instrument is introduced through an opening of the instrument seal in a substantially perpendicular orientation with respect to the instrument seal. In certain situations, when a surgical instrument is manipulated by a clinician during a surgical procedure, some gasses and fluids escape through the opening around the instrument seal. More particularly, when the surgical instrument is manipulated in an off-axis position, i.e., where the surgical instrument is not in a substantially perpendicular orientation, an elongated opening around the surgical instrument is created. This elongated opening is often referred to as a “cat-eye” effect. The “cat-eye” effect allows unintended gases and fluids to enter and/or exit through the opening of the instrument seal.

Accordingly, a continuing need exist in the medical arts for a compliant instrument seal that can maintain an effective seal during off-axis manipulation of the surgical instrument during a surgical procedure.

### SUMMARY

The present disclosure relates to a contoured instrument seal for arthroscopic, laparoscopic and endoscopic surgery. The contoured instrument seal includes a body, an annular inner edge, an annular outer edge, and one or more raised concentric rings. The body includes a top surface and a bottom surface, which may be formed of a resilient material. The surface of the resilient body may have a reduced thickness on at least one side of the raised concentric ring. The annular inner edge defines an opening that is adapted to receive a surgical instrument. The annular outer edge may be adapted to mechanically mount to an inner portion of a seal housing. The annular inner edge may define an area of increased thickness. The area of increased thickness and the raised concentric ring define a first area of reduced thickness therebetween. The raised concentric ring is defined on a surface of the resilient body between the annular inner edge and the annular outer edge.

In embodiments, the one or more raised concentric rings may include a spiral configuration such that a first end of the spiral configuration is approximate the annular inner edge and a second end of the spiral configuration is approximate the annular outer edge. In addition, the raised concentric rings may be defined on the top surface, bottom surface, or both top and bottom surfaces of the resilient body. The raised concentric rings on the top and bottom surfaces of the resilient body may be vertically aligned or vertically offset from each other.

The present disclosure also relates to a surgical portal assembly for use during a surgical procedure. The surgical portal assembly includes a seal housing, a sleeve, and a contoured instrument seal, as described above. The sleeve is mountable to the seal housing and has an internal longitudinal passage adapted to provide access to underlying tissue. The contoured instrument seal is mechanically coupled within the seal housing.

### BRIEF DESCRIPTION OF THE DRAWINGS

Various embodiments of the present disclosure are described herein with reference to the drawings wherein:

FIG. 1 is a perspective view of a surgical portal apparatus including a contoured compliant instrument seal in accordance with an embodiment of the present disclosure;

FIG. 2A is a perspective view of the contoured compliant instrument seal of FIG. 1;

FIG. 2B is a side, cross-sectional view illustrating the contoured compliant instrument seal of FIG. 2A including a body having a plurality of raised concentric rings on top and bottom surfaces;

FIG. 2C is a side, cross-sectional view illustrating another embodiment of a contoured compliant instrument seal including a body having a raised concentric ring on a top surface;

FIG. 3A is a perspective view of another embodiment of a contoured compliant instrument seal including a body having a spiral-like concentric ring on a top surface;

FIG. 3B is a side, cross-sectional view illustrating the contoured compliant instrument seal of FIG. 3A including a body having a spiral-like concentric rings on top and bottom surfaces;

FIG. 3C is a side, cross-sectional view of another embodiment of a contoured compliant instrument seal including a body having a spiral-like concentric ring on a top surface;

FIG. 4A is a perspective view of yet another embodiment of a contoured compliant instrument seal in accordance with the present disclosure;

FIG. 4B is a side, cross-sectional view illustrating the contoured compliant instrument seal of FIG. 4A including a body having raised concentric rings on top and bottom surfaces; and

FIG. 4C is a side, cross-sectional view illustrating another embodiment of a contoured compliant instrument seal including a body having a raised concentric ring on a top surface.

### DETAILED DESCRIPTION

The portal apparatus of the present disclosure provides for the introduction of various types of instrumentation to a surgical site within a body cavity or joint and incorporates an instrument seal that is adapted to maintain a substantially fluid-tight interface about the instrumentation by minimizing the entry and exit of gases and/or fluids to and from the body cavity or joint.

Examples of instrumentation include, but are not limited to, clip appliers, graspers, dissectors, retractors, staplers, laser probes, photographic devices, endoscopes, laparoscopes, tubes, and the like. Such instrumentation will collectively be referred to as “surgical instruments,” “instrumentation,” or “surgical objects” which also may include the hand of a clinician.

The portal assembly may be any suitable cannula assembly used in arthroscopic, laparoscopic or endoscopic procedures. The portal assembly may also be adapted to receive the hand of a clinician during, e.g., a minimally invasive laparoscopic hand assisted procedure.

In the following description, as is traditional, the term “proximal” or “trailing” refers to the portion of the device closer to the clinician while the term “distal” or “leading” refers to the portion of the device further from the clinician.

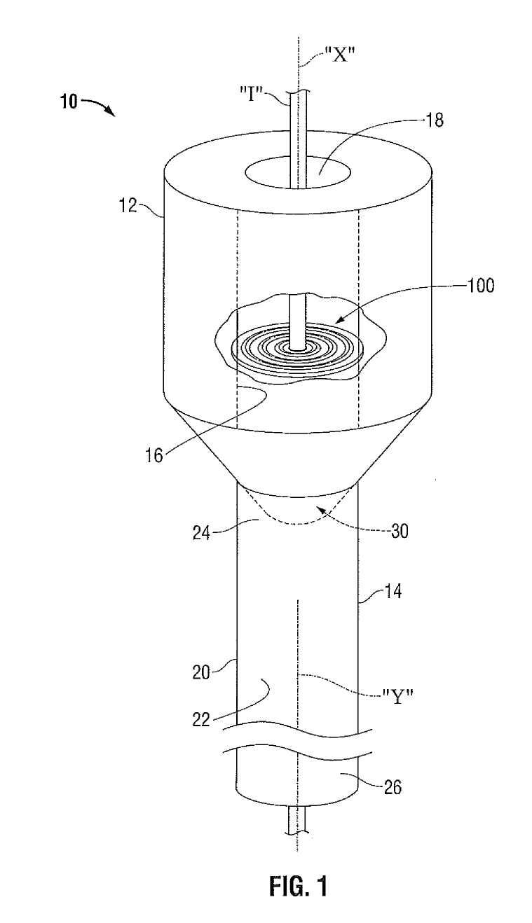

Referring now to the drawings, in which like reference numerals identify identical or substantially similar parts throughout the several views, FIG. 1 illustrates an exemplary embodiment of the portal apparatus 10 in accordance with the principles of the present disclosure. Portal apparatus 10 may be an arthroscopic, laparoscopic or endoscopic cannula assembly utilized in conjunction with a surgical procedure where a body cavity is insufflated with a suitable gas, e.g., CO₂, to raise a wall of the body cavity from the internal organs therein or a joint is provided with a suitable fluid, e.g., saline solution, to provide access to joint structures. The cannula assembly may be used with an obturator assembly (not shown) which is a sharp pointed instrument positionable within the passageway of the cannula assembly. The obturator assembly is utilized to penetrate the body wall and is then subsequently removed from the cannula assembly to permit introduction of the surgical instrumentation utilized to perform the procedure.

Portal apparatus 10 includes portal housing 12 and elongated portal member 14 extending from the portal housing 12. Portal housing 12 may include multiple housing segments connected to each other via conventional means or may be a single component integrally or monolithically formed. Portal housing 12 has an inner housing wall 16 defining a housing passage 18 coaxially arranged about a longitudinal housing axis “X” extending through the portal housing 12. Inner housing wall 16 is dimensioned to accommodate an instrument seal, for example, a contoured compliant instrument seal 100, as shown in FIG. 1. Inner housing wall 16 and instrument seal 100 cooperate with each other to receive a surgical object or instrument “I” and laterally confine instrument “I” within portal housing 12. Inner housing wall 16 may be generally circular in cross-section or may assume other cross-sectional shapes.

Portal member 14 may be a sleeve member defining a longitudinal portal axis “Y” extending along the length of the portal member 14. Longitudinal portal axis “Y” of portal member 14 may be in general longitudinal alignment with longitudinal housing axis “X” of portal housing 12. Portal member 14 includes outer sleeve wall 20 defining an internal longitudinal opening 22 extending from proximal or trailing end 24 through distal or leading end 26 of the portal member 14. Longitudinal opening 22 of portal member 14 is in general longitudinal alignment with central housing passage 18 of portal housing 12 to define a common longitudinal passageway 18, 22 through portal apparatus 10 for passage of the surgical object. Portal member 14 may be a separate component connected to portal housing 12 or may be monolithically formed with the portal housing 12. Portal member 14 and portal housing 12 may be releasably connected through a variety of mechanisms including, for example, but not limited to, a bayonet lock, threaded connection, or the like.

Portal member 14 may be formed of stainless steel or another rigid material such as a polymeric material or the like. Portal member 14 may be clear or opaque. The diameter of portal member 14 may vary, but typically ranges from about 3 to about 15 mm when used in laparoscopic or endoscopic procedures. If used in a hand assisted minimally invasive approach, the diameter of portal member may be substantially greater than 15 mm.

FIG. 1 illustrates an exemplary embodiment of the presently disclosed contoured instrument seal 100 and is situated in portal assembly 10. It is envisioned that the presently disclosed embodiments of the contoured instrument seals may be adapted to be situated in any suitable surgical assembly or apparatus, e.g., cannula assembly, where an instrument seal is necessary.

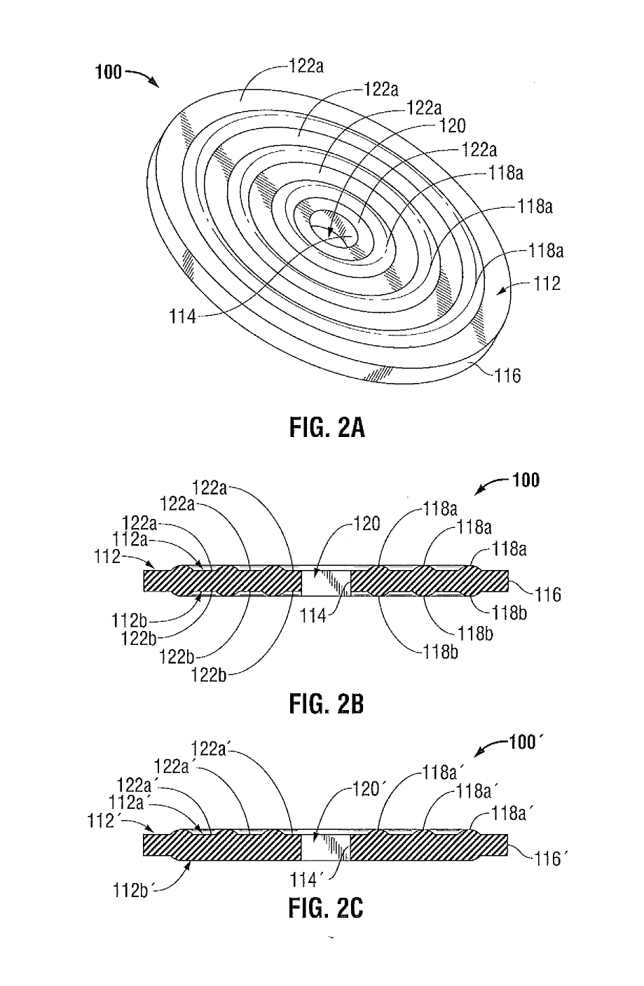

FIGS. 2A-2B illustrate one embodiment of the presently disclosed contoured instrument seal and is shown generally as 100. Contoured instrument seal 100 includes a body 112, which includes an annular inner edge 114, an annular outer edge 116, and one or more raised concentric rings 118a (“raised” is used herein to refer to a height which is relatively greater than the height of some other portion of the instrument seal, it being acknowledged that a seal that has grooves or channels cut or otherwise formed into its surface also has “raised” rings between the grooves or channels). Body 112 is formed of any suitable resilient material, for example, an elastomeric material, and includes a top surface 112a and a bottom surface 112b. Annular inner edge 114 defines an opening 120 that is adapted to receive a surgical instrument “I” during a surgical procedure. Annular outer edge 116 may be adapted to mechanically mount to inner housing wall 16 of portal housing 12.

One or more raised concentric rings 118a are formed on top surface 112a of resilient body 112 and disposed between annular inner edge 114 and annular outer edge 116. Each raised concentric ring 118a is formed on top surface 112a and is spaced from an adjacent concentric ring(s). For example, a raised inner concentric ring 118a having a smaller diameter is positioned towards annular inner edge 114 and a raised outer concentric ring 118a having a larger diameter is positioned towards annular outer edge 116. As illustrated, a raised central concentric ring 118a may be formed between the inner and outer rings.

Raised concentric rings 118a may protrude above surface 112a of resilient body 112 to thereby provide a greater thickness to resilient body 112 at spaced locations on body 112. In this manner, raised concentric rings 118a may provide structural rigidity to resilient body 112 of compliant instrument seal 100, thus reducing the so-called “cat eye” effect when an instrument “I” is laterally manipulated within opening 120 of body 112 (i.e., in an off-axis position).

In one embodiment, raised concentric rings 118a may be spaced apart from each other by any suitable distance to define a substantially flat surface 122a between adjacent rings 118a and/or between rings 118a and inner and outer edges 114 and 116. Surfaces 122a of body 112 each define an area of reduced thickness when compared to the thickness of raised concentric rings 118a of body 112. In some embodiments, the reduced thickness of surface area 122a may be further reduced, by any suitable cutting or forming technique, to define grooves. Each of flat surfaces 122a is positioned and configured on body 112 to improve compliance (i.e., flexibility) of instrument seal 100 when a surgical instrument “I” is inserted through opening 120 and moved laterally. As such, raised concentric rings 118a provide structural rigidity throughout compliant instrument seal 100 to minimize “cat-eye” effect when surgical instrument “I” is laterally manipulated during surgical procedures.

In one embodiment, as best shown in FIG. 2B, both top surface 112a and bottom surface 112b include raised concentric rings 118a, 118b. Similarly to top surface 112a, bottom surface 112b defines one or more raised concentric rings 118b and flat surfaces 122b between annular inner edge 114 and annular outer edge 116.

Raised concentric rings 118a, 118b on top and bottom surfaces 112a and 112b of resilient body 112 may be vertically aligned such that raised concentric rings 118a, 118b are mirror-imaged (i.e., opposite) from each other. In this manner, the overall thickness of concentric rings 118a, 118b of instrument seal 100 is approximately doubled, thus providing more structural rigidity to selected areas of body 112 of instrument seal 100 during surgical procedures. Alternatively, as shown in FIG. 2C, raised concentric rings 118a′ and 118b′ may only be provided on a top or bottom surface 112a′ or 112b′ of body 112′. In the exemplary embodiment of FIG. 2C, contoured seal 100′ includes raised concentric rings 118a′, which are formed on top surface 112a′ of body 112′.

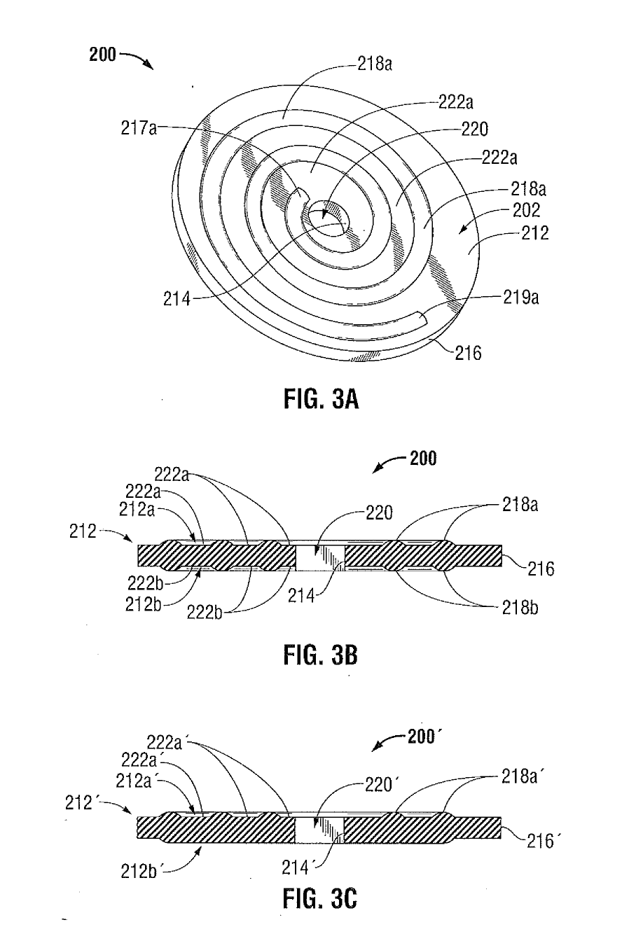

FIGS. 3A-3B illustrate another embodiment of the presently disclosed contoured instrument seal shown generally as 200. Similarly to instrument seal 100, contoured instrument seal 200 includes a body 212, an annular inner edge 214, and an annular outer edge 216. However, contoured instrument seal 200 includes a raised concentric ring 218a that is formed into a spiral-like configuration having a first end 217a that continuously coils (i.e., spirals) to a second end 219a. First end 217a of raised spiral concentric ring 218a is positioned adjacent annular inner edge 214 and second end 219a of raised spiral concentric ring 218a is positioned adjacent annular outer edge 216.

Body 212 is formed of any suitable resilient material, for example, an elastomeric material, and includes a top surface 212a and a bottom surface 212b. Annular inner edge 214 defines an opening 220 that is adapted to receive a surgical instrument “I” during a surgical procedure. Annular outer edge 216 may be adapted to mechanically mount to inner housing wall 16 of portal housing 12, as shown in FIG. 1.

Raised spiral concentric ring 218a is formed on top surface 212a of resilient body 212 between annular inner edge 214 and annular outer edge 216 and may protrude above surface 212a of resilient body 212, thereby providing a greater thickness to resilient body 212 in the area of spiral ring 218a. In this manner, raised spiral concentric ring 218a provides structural rigidity to surface 212 of compliant instrument seal 200, thus reducing the so-called “cat eye” effect when an instrument “I” is laterally manipulated within opening 220.

In one embodiment, raised spiral concentric ring 218a may be formed in a coil-like configuration such that each continuous ring is spaced apart by any suitable distance to define a spiral flat surface 222a. Surface 222a of body 212 has a reduced thickness as compared to raised concentric rings 218a of body 212. In some embodiments, the thickness of surface area 222a may be further reduced, by any suitable cutting or forming technique, to define grooves. As described above, reduced thickness area 222a is configured to improve compliance (i.e., flexibility) of instrument seal 200 when a surgical instrument “I” is inserted through opening 220 and moved laterally, while raised concentric rings 218a provide structural rigidity throughout compliant instrument seal 200.

In one embodiment, as best shown in FIG. 3B, both top surface 212a and bottom surface 212b include a raised spiral concentric ring 218a, 218b. Similarly to top surface 212a, bottom surface 212b includes raised concentric ring 218b that is formed into a spiral-like configuration having a first end 217b that coils (i.e., spirals) to a second end 219b. First end 217b of raised spiral concentric ring 218b is positioned adjacent annular inner edge 214 of body 212 and second end 219b of raised spiral concentric ring 218b is positioned adjacent annular outer edge 216.

In one embodiment, as shown in FIG. 3B, raised concentric spiral rings 218a, 218b of top and bottom surfaces 212a and 212b of resilient body 212 may be vertically offset such that they are misaligned from each other. In this manner, concentric spiral rings 218a, 218b of instrument seal 200 provide structural rigidity on scattered portions of resilient body 212, thus distributing the structural rigidity throughout instrument seal 200.

Alternatively, in another embodiment, raised concentric spiral rings 218a, 218b of top and bottom surfaces 212a and 212b of resilient body 212 may be vertically aligned such that they are mirror imaged (i.e., opposite) from each other. In this manner, the overall thickness of concentric spiral rings 218a, 218b of instrument seal 200 is approximately doubled, thus providing more structural rigidity to selected areas of instrument seal 200. Alternatively, as shown in FIG. 3C, raised concentric spiral rings 218a′ and 218b′ may only be provided on a top or bottom surface 212a′ or 212b′ of body 212′. In the exemplary embodiment of FIG. 3C, contoured seal 200′ includes raised concentric spiral rings 218a′, which are formed on top surface 212a′ of body 212′.

The spiral arrangements shown, for example, in the embodiments of FIGS. 3A-3C may have additional advantages in that fluid that is brought into contact with the instrument seal 200 is more readily moved away from the center thereof. Specifically, as an instrument is inserted through opening 220, fluid that is carried by the instrument, e.g., sterilization agents, blood, saline solution, other bodily fluids, etc., may collect in the center region of the seal 200 near the opening 220. With respect to laparoscopic surgery, it is generally undesirable for these fluids to collect here, since they are more likely to leak, or be ejected by the insufflation pressure inside the body cavity, through the opening 200 (as previously noted, it is typical in arthroscopic surgery that fluid, such as saline fluid, is employed within a surgical space rather than insufflation gas). In addition, fluids or other matter that collect here may foul an endoscope inserted therethrough, thereby reducing the ability of the endoscope to visualize the body cavity during surgery and requiring the endoscope to be removed and re-cleaned repeatedly during a surgical procedure. The spiral arrangement described above and shown, for example, in FIGS. 3A-3C may decrease the likelihood of this occurring by providing a continuous and lengthy channel, formed by the bottom surfaces 222a, 222b etc., into which fluid that collects near the opening 220 may be conveyed and carried away from the opening 220. The conveyance of the fluid along this channel may occur by, e.g., capillary action, gravity etc.

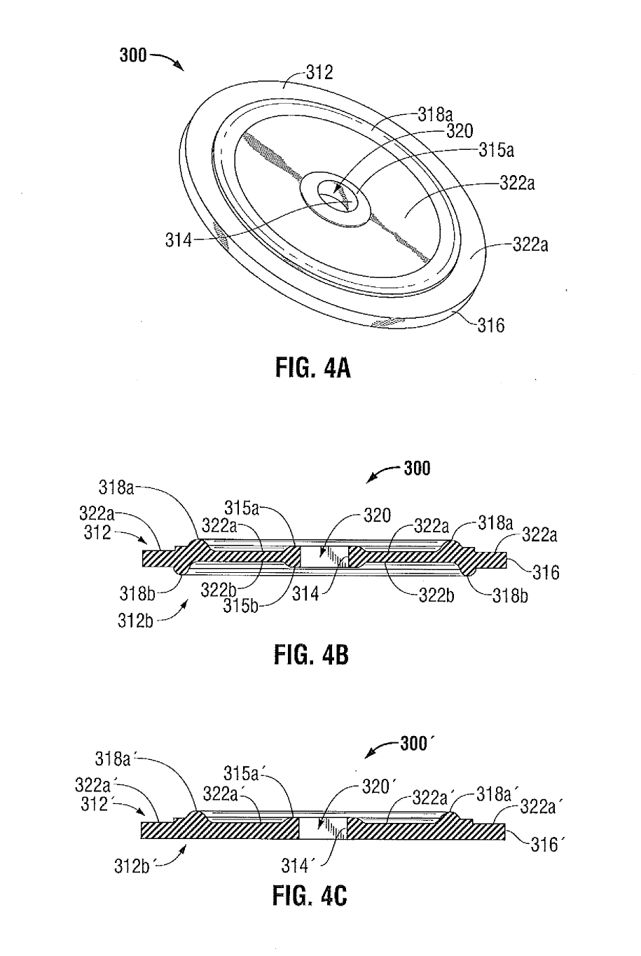

FIGS. 4A-4B illustrate an additional embodiment of the presently disclosed contoured instrument seal shown generally as 300. Contoured instrument seal 300 includes a body 312, an annular inner edge 314, an annular outer edge 316, and a raised concentric ring 318a. Body 312 is formed of any suitable resilient material, for example, an elastomeric material, and includes a top surface 312a and a bottom surface 312b. Similarly to contoured instrument seals 100 and 200 described above, annular inner edge 314 defines an opening 320 that is adapted to receive a surgical instrument “I.” However, annular inner edge 314 defines an area of increased thickness 315a. The area of increased thickness 315a and raised concentric ring 318a define a first area of reduced thickness 322a therebetween. By providing support around annular inner edge 314 when an instrument “I” is inserted and moved laterally, the area of increased thickness 322a and raised concentric rings 318a are configured to substantially reduce the so-called “cat-eye effect,” whereas the area of reduced thickness provides a degree of pliability to body 312 to facilitate lateral movement of an instrument “I.” As discussed above, annular outer edge 316 of body 312 may be adapted to mechanically mount to inner housing wall 16 of portal housing 12.

Raised concentric ring 318a is formed on top surface 312a of resilient body 312 between annular inner edge 314 and annular outer edge 316. Raised concentric ring 318a may be defined on any portion of top surface 312a thereby changing the diameter of raised concentric ring 318a depending on the position of raised concentric ring 318a on top surface 312a. For example, raised concentric ring 318a may have a small diameter when raised concentric ring 318a is positioned on a portion of surface 312a adjacent annular inner edge 314. Additionally or alternatively, raised concentric ring 318a may have a large diameter when raised concentric ring 318a is positioned adjacent annular outer edge 316.

Raised concentric ring 318a may protrude above surface 312a of resilient body 312, thereby providing a greater thickness to resilient body 312. As described above, raised concentric ring 318a, in this manner, provides structural rigidity to selected areas of body 312 of compliant instrument seal 300, thus reducing the so-called “cat eye” effect when an instrument “I” is laterally manipulated within opening 320.

The area approximate raised concentric ring 318a (i.e., on either side of raised concentric ring 318a) defines a flat surface 322a of surface 312a. Body 312 defining surface 322a has a reduced thickness when compared to a thickness of raised concentric rings 318a of body 312. In embodiments, the thickness of surface area 322a may be further reduced, by any suitable cutting or forming technique, to define a large cavity. Reduced thickness area 322a is configured to improve compliance (i.e., flexibility) of instrument seal 300 when a surgical instrument “I” is inserted through opening 320 and moved laterally, while the raised concentric rings provide structural rigidity to compliant instrument seal 300.

In one embodiment, as best shown in FIG. 4B, both top surface 312a and bottom surface 312b include raised concentric rings 318a, 318b. Similarly to top surface 312a, bottom surface 312b defines a raised concentric ring 318b and flat surface areas 322b between annular inner edge 314 and annular outer edge 316. Alternatively, as shown in FIG. 4C, raised concentric rings 318a′ and 318b′ may only be provided on a top or bottom surface 312a′ or 312b′ of body 312′. In the exemplary embodiment of FIG. 4C, contoured seal 300′ includes raised concentric rings 318a′, which are formed on top surface 312a′ of body 312′.

As best shown in FIG. 4B, raised concentric rings 318a, 318b of top and bottom surfaces 312a and 312b of resilient body 312 may be vertically offset such that raised concentric rings 318a, 318b are misaligned from each other. In this manner, concentric rings 318a, 318b of instrument seal 300 provide structural rigidity on different portions of resilient body 312, thus distributing the structural rigidity throughout instrument seal 300 during surgical procedures.

Alternatively, in another embodiment, raised concentric rings 318a, 318b on top and bottom surfaces 312a and 312b of resilient body 312 may be vertically aligned such that they are mirror imaged (i.e., opposite) from each other. In this manner, the overall thickness of concentric rings 318a, 318b of instrument seal 300 is approximately doubled, thus providing more structural rigidness to the instrument seal 300 during surgical procedures.

The materials of fabrication of instrument seals 100, 200, and 300 may include a suitable elastomeric material, for example, but not limited to, isoprene or natural rubber. In the disclosed embodiments, instrument seals 100, 200, and 300 may be coated with a hydrophilic coating to facilitate passage of the surgical instrument “I.”

In one embodiment, portal housing 12 may also include a zero closure valve 30 disposed in mechanical cooperation within housing 12 (as shown in FIG. 1). Zero closure valve 30 may be, for example, but not limited to, a duckbill valve, slit valve, trumpet valve or the like. Zero closure valve 30 is adapted to provide a substantially fluid-tight seal in absence of a surgical object.

During use, a surgical object or surgical instrument “I” (as shown in FIG. 1) is introduced through the housing passage 18 and through any one of the embodied compliant seals 100, 200, or 300 described above. More specifically, the surgical instrument “I” is advanced through seal 100, 200, and 300 and longitudinal opening 18 of portal member 12 whereby seal passages 120, 220, and 320 cooperate to establish a seal about the surgical instrument “I” while a clinician performs a surgical procedure.

While several embodiments of the disclosure have been shown in the drawings and/or discussed herein, it is not intended that the disclosure be limited thereto, as it is intended that the disclosure be as broad in scope as the art will allow and that the specification be read likewise. Therefore, the above description should not be construed as limiting, but merely as exemplifications of particular embodiments. Those skilled in the art will envision other modifications within the scope and spirit of the claims appended hereto.

What is claimed is:

1. An instrument seal for arthroscopic, laparoscopic and endoscopic surgery, the contoured instrument seal comprising:
a body formed of a resilient material and having an annular inner edge defining an opening adapted to receive a surgical instrument and an annular outer edge spaced from the annular inner edge; and
a plurality of raised concentric rings having a spiral shape and defined on a surface of the body of the contoured instrument seal between the annular inner edge and the annular outer edge of the body.

2. A contoured instrument seal, according to claim 1, wherein the plurality of raised concentric rings has a shape configured to convey fluid away from the annular inner edge of the body and towards the annular outer edge of the body.

3. The contoured instrument seal according to claim 1, wherein the annular inner edge defines an area of increased thickness.

4. The contoured instrument seal according to claim 1, wherein the area of increased thickness and the plurality raised concentric rings define an area of reduced thickness therebetween.

5. The contoured instrument seal according to claim 1, wherein the resilient body has a reduced thickness on at least one side of the plurality raised concentric rings.

6. The surgical portal assembly according to claim 1, wherein the spiral configuration has a first end at the annular inner edge and a second end at the annular outer edge.

7. The contoured instrument seal according to claim 1, wherein the resilient body includes a top surface and a bottom surface.

8. The contoured instrument seal according to claim 7, wherein the plurality of raised concentric rings are defined on the top surface of the resilient body.

9. The contoured instrument seal according to claim 7, wherein the plurality of raised concentric rings of the resilient body are defined on the top surface and the bottom surface of the resilient body.

10. The contoured instrument seal according to claim 9, wherein the plurality of raised concentric rings are defined on the top surface of the resilient body and a plurality of raised concentric rings are defined on the bottom surface of the resilient body.

11. The contoured instrument seal according to claim 10, wherein the plurality of raised concentric rings on the top surface of the resilient body and the plurality of raised concentric rings on the bottom surface of the resilient body are vertically aligned.

12. The contoured instrument seal according to claim 10, wherein the plurality of raised concentric rings on the top surface of the resilient body and the plurality of raised concentric rings of the bottom surface of the resilient body are vertically offset from each other.
